(19) United States
(12) Patent Application Publication
(10) Pub. No.: US 2010/0076492 A1
(43) Pub. Date: Mar. 25, 2010

(54) SPINOUS PROCESS SPACER IMPLANT AND TECHNIQUE

(75) Inventors: Kenneth D. Warner, Wilton, CT (US); Steven B. Goldstein, Plainview, NY (US)

Correspondence Address: KNOBBE MARTENS OLSON & BEAR LLP, 2040 MAIN STREET, FOURTEENTH FLOOR, IRVINE, CA, 92614, US

(73) Assignee: EVOLUTION SPINE TECHNOLOGIES, LLC., Wilton, CT (US)

(21) Appl. No.: 12/629,806

(22) Filed: Dec. 2, 2009

Related U.S. Application Data
(62) Division of application No. 11/308,767, filed on May 1, 2006, now abandoned.
(60) Provisional application No. 60/676,538, filed on May 2, 2005.

(51) Int. Cl. A61B 17/88

(52) U.S. Cl. 606/279

(57) ABSTRACT

A spinous process spacer implant and method of use are provided. The implant can be configured to be placed between adjacent spinous processes. The implant can comprise at least one pair of notches that can be positioned such that portions of the adjacent spinous processes are engaged therewithin to maintain a size of a desired space between the adjacent spinous processes.

### CROSS-REFERENCE TO RELATED APPLICATIONS

This application is a divisional of U.S. application Ser. No. 11/308,767, filed May 1, 2006, which claims the benefit of U.S. Provisional Application No. 60/676,538, filed on May 2, 2005, the entireties of each of which are hereby incorporated herein by reference.

### BACKGROUND

1. Field of the Inventions

This invention generally relates to a device and method for the treatment of spinal stenosis.

2. Description of the Related Art

Spinal stenosis is a narrowing of the spinal canal. While this in itself does not necessarily cause symptoms, swelling and nerve inflammation results when the narrowing leads to compression of the spinal cord and nerve roots.

While spinal stenosis can be found in any part of the spine, the lumbar and cervical areas are most commonly affected. Patients with lumber spinal stenosis may feel pain, weakness, or numbness in the lower extremities. Symptoms often increase when walking short distances and decrease when the patient sits, bends forward or lies down. Although some people are born with spinal stenosis, it generally occurs as the gradual result of “wear and tear” on the spine during everyday activities, primarily affecting people over 50 years of age.

Non-surgical treatments of spinal stenosis include medication, steroid injections, and physical therapy. While surgical options are available, they are invasive. Due to the inherent risks involved with such procedures, surgery is usually considered only after other non-invasive procedures have failed.

Published application number 2001/0039452 by Zucherman discloses a spinal distraction implant that alleviates pain associated with spinal stenosis by expanding the volume in the spinal canal or neural foramen. In the Zucherman device, a body portion is adapted to seat between the adjacent spinous processes while a wing portion is adapted to prevent lateral movement of the body portion, thereby holding it in place between the adjacent spinous processes.

Although the Zucherman device achieves spinal distraction, it nonetheless presents some limitations. It is a non-biological, multi-piece design, subject to wear and implantation complexity. Furthermore, the expansive geometry of the device may not lend itself to minimally invasive surgical techniques seeking to conserve muscle mass and soft tissue in the regions adjacent the spinous processes.

### SUMMARY

A spinous process spacer device for surgical implantation between the spinous processes of adjacent upper and lower vertebrae is disclosed. The spacer device maintains a desired space between the adjacent spinous processes. It comprises a tubular member having an axis, a length, an axial lumen coextensive with the length, an outer diameter, an upper end and a lower end. The upper and lower ends each have a pair of diametrically opposed notches (cut outs) along the outer diameter of the spacer device. The pair of diametrically opposed notches in the upper end is aligned with the pair of diametrically opposed notches in the lower end. When properly positioned, the pairs of diametrically opposed notches are dimensioned to receive a portion of the spinous processes, thereby maintaining the desired space between adjacent spinal processes.

The features of the invention believed to be novel are set forth with particularity in the appended claims. However the invention itself, both as to organization and method of operation, together with further objects and advantages thereof may be best understood by reference to the following description taken in conjunction with the accompanying drawings.

### BRIEF DESCRIPTION OF THE FIGURES

The abovementioned and other features of the inventions disclosed herein are described below with reference to the drawings of the preferred embodiments. The illustrated embodiments are intended to illustrate, but not to limit the inventions. The drawings contain the following figures:

FIG. 1 illustrates a spinal column having a collapsed disc and stenotic central canal.

FIG. 2 illustrates the spinous process spacer implant of the present invention.

FIG. 3a illustrates the final configuration of spinous process spacer positioned between adjacent spinous processes. FIG. 3b is an enhancement indicating the engagement of the spinous processes with the spinous process cutout portion of the implant.

FIG. 4 illustrates the spinous process spacer positioned and constrained with optional cables.

FIG. 5 illustrates the introduction of successively larger diameter dilators until the appropriate distraction is achieved.

FIG. 6 illustrates the insertion and placement of the rotation cannula over the last dilator used.

FIG. 7 illustrates the position of the rotation cannula between adjacent spinous processes after the dilators have been removed.

FIG. 8 illustrates the insertion and initial placement of the implant with the rotation instrument.

FIG. 9 illustrates the configuration upon rotating the implant 90 degrees using the rotation instrument.

FIG. 10 illustrates the final configuration of the implant upon removal of the cannula and rotation instrument.

FIG. 11 illustrates the empty rotation cannula.

FIG. 12 illustrates the rotation cannula with the implant inserted into the extensions for the lateral spinous process stabilizers.

FIG. 13 illustrates the rotation instrument.

FIG. 14 illustrates the rotation instrument with the implant inserted between its prongs.

FIG. 15 illustrates the rasping device.

### DESCRIPTION OF THE NUMERALS USED IN THE FIGURES

10—spinal cord
11—vertebral body
12—spinous process
13—normal interspinous process space
14—collapsed interspinous process space
15—stenotic central canal
16—normal disc
17—collapsed disc
18—anterior side
19—posterior side
20—implant, spinous process spacer
21—tubular member
22—axial conduit
23—spinous process cutout, notch
24—lateral spinous process stabilizers
25—holes for securement means
26—desired interspinous process spatial distance
40—means for securement to spinous processes
50—dilators
60—rotation cannula
80—rotation instrument
110—extension for lateral spinous process stabilizers
111—rotation and removal grooves
112—tapered leading edge of the extension
113—open trailing edge of the extension
130—prong
131—saddle
150—trial rasp
151—bullet shaped leading edge
152—implant shaped body
153—stem
154—handle
155—rasping surface

### DETAILED DESCRIPTION

The term “allograft”, as used herein, is intended to mean a graft taken from a different individual of the same species.

The term “sagittal plane”, as used herein, is the plane which splits the body into left and right segments. The mid-sagittal, or median plane splits the body into equal left and right halves.

The term “coronal plane”, as used herein, is the plane that separates the body into anterior and posterior (front and back) segments. The coronal plane is perpendicular to the sagittal plane.

The term “posterior process fusion”, as used herein, describes the fusion of adjacent spinous processes firstly to the spinous process spacer implant, and eventually to each other via the growth of tissue through the axial conduit of the implant.

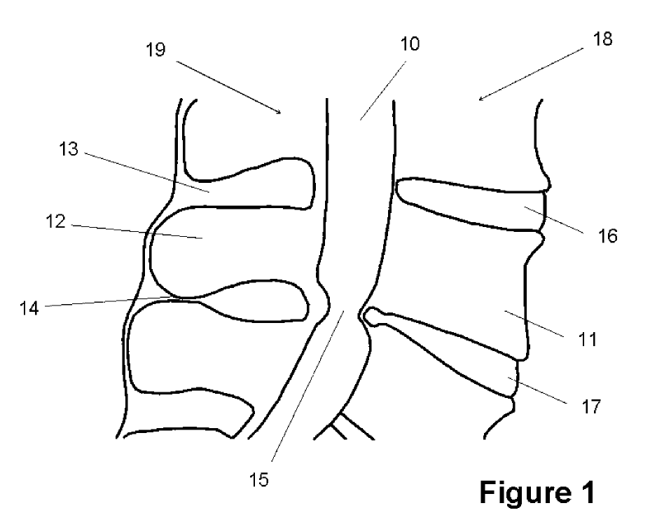

The function of the spinous process spacer (20) can be understood by appreciating the problem illustrated in FIG. 1. A cross section of the spinal cord (10) in the mid-sagittal plane is shown with the vertebral body (11) on the right and the spinous process (12) on the left. A normal disc (16) above (or cephalad to) the vertebral body (11) is shown pairing with a normal interspinous process space (13). In the same manner, a collapsed disc (17) pairs with a collapsed interspinous process space (14). This unbalanced arrangement results in one or more types of narrowing such as the stenotic central canal shown at (15). The spinous process spacer (20) spreads the spinous processes (12) adjacent to the collapsed interspinous process space (14) apart, thereby restoring anatomical alignment of the anterior and posterior spinal anatomy and alleviating the narrowing of nerve pathways that may have been generating severe pain and loss of function.

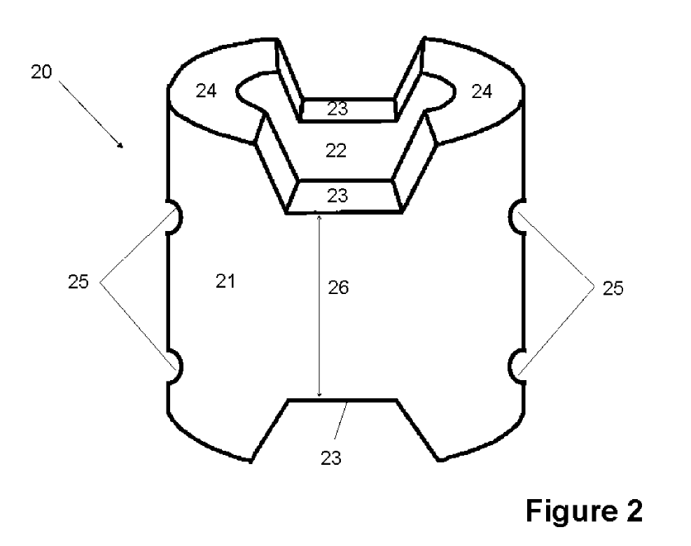

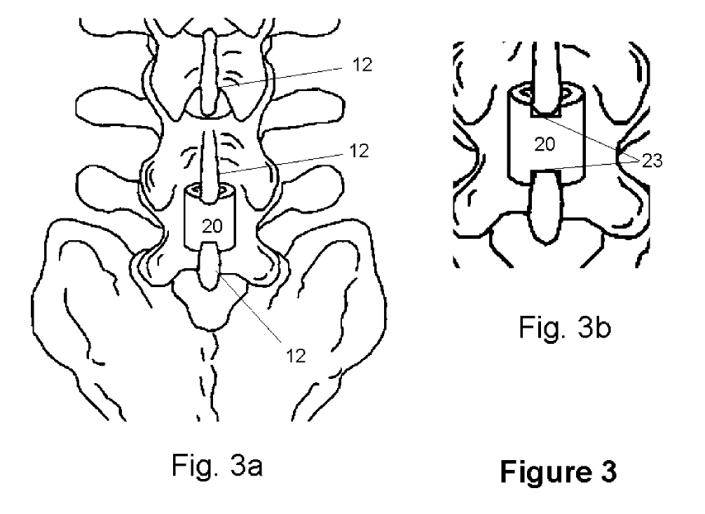

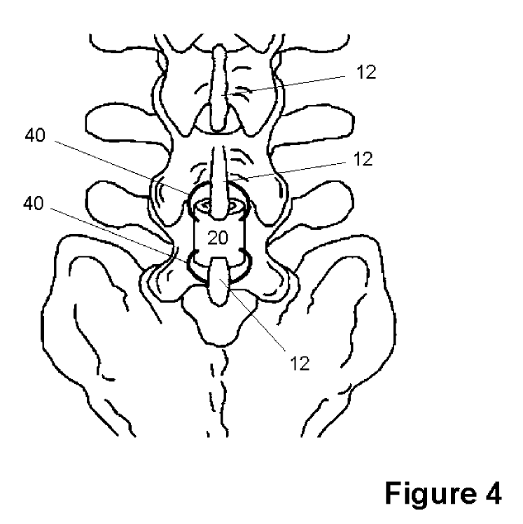

The spinous process spacer (20) of the present invention is shown in FIG. 2. Basically a tubular member (21) having an axial conduit (22), the desired interspinous process space (26) is defined by the depth of diametrically opposed spinous process cutouts, or, more simply termed “notches” (23), cut out of each end. The spinous processes (12) seat in these notches (23) when the spinous process spacer (20) is deployed in its final position as illustrated in FIGS. 3-4. The remaining tubular sections flanking the notches (23) function as lateral spinous process stabilizers (24). Optional holes (25) for mechanical attachment (40) to the adjacent spinous processes (12) are provided as well. Attachment can be effected by suturing, cabling or other suitable means as is indicated in FIG. 4.

The tubular geometry of the spinous process spacer (20) not only serves to strengthen the spacer in the axial direction but also provides more stabilization against unintended rotation than the substantially flat “H” shaped designs of the prior art. Moreover, the axial conduit (22) offers a fillable space for bone growth-promoting materials. Finally, the spinous process spacer (20) can be made of allograft or other suitable biological material to further promote integration of the spinous process spacer (20) into surrounding tissue.

There are many techniques suitable for deployment of the spinal process spacer (20), the choice of which is dependent upon individual circumstances. Basically, the following steps must be executed. The collapsed interspinous process space (14) must be adequately distracted. An appropriately sized spinous process spacer implant (20) must be secured in an implant holder. The holder and implant must be placed within the distracted space so that the axis of the spinous process spacer implant (20) is parallel to the adjacent spinous processes. Finally the spinous process spacer implant (20) is rotated 90 degrees in the mid-sagittal plane so that its axis is now perpendicular to the spinous processes (12), and positioned so that the spinous processes (12) rest within the notches or, alternatively, the spinous process cutouts (23), and engagement is effected. A major benefit of this technique is that it gains access to the spinous process space via a lateral incision in the spinous process ligament. This preserves more of the spinous process ligament than a direct posterior approach.

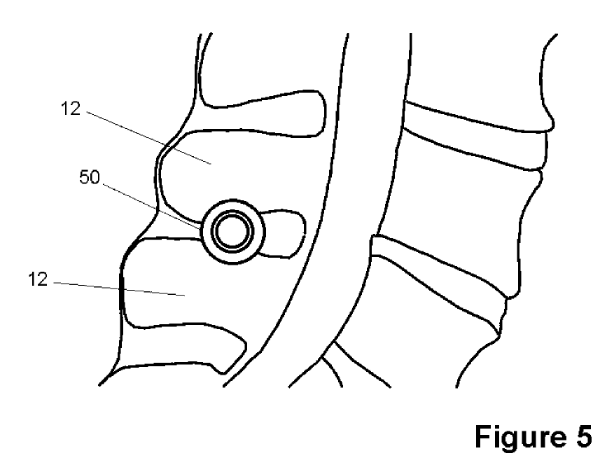

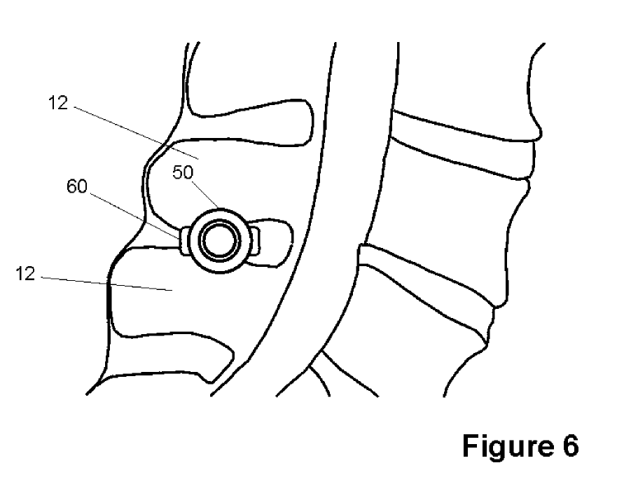

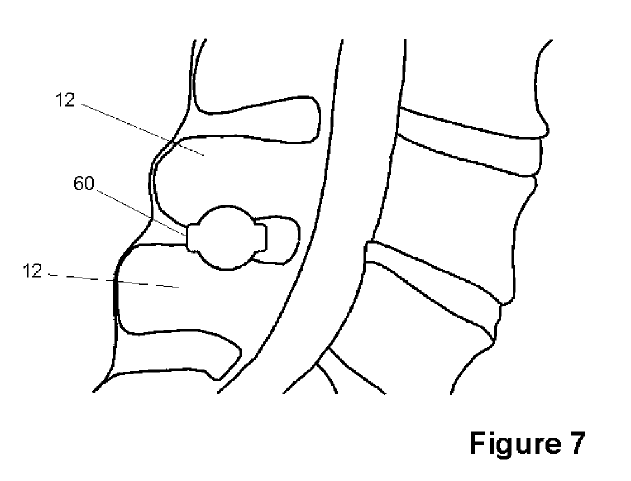

A more detailed description of such a method is illustrated in the remaining FIGS. 5-15. FIG. 5 illustrates a cross section of the mid-sagittal plane wherein dilators (50) have been placed between adjacent spinous processes (12). A rotation cannula (60) is placed over the largest dilator as shown in FIG. 6. The rotation cannula (60) is described in more detail in later paragraphs, but for the present discussion, suffice it to say that the rotation cannula (60) effects the above outlined steps of providing an implant holder and facilitating the required 90 degree rotation. FIG. 7 illustrates the position of the rotation cannula between adjacent spinous processes after the dilators have been removed.

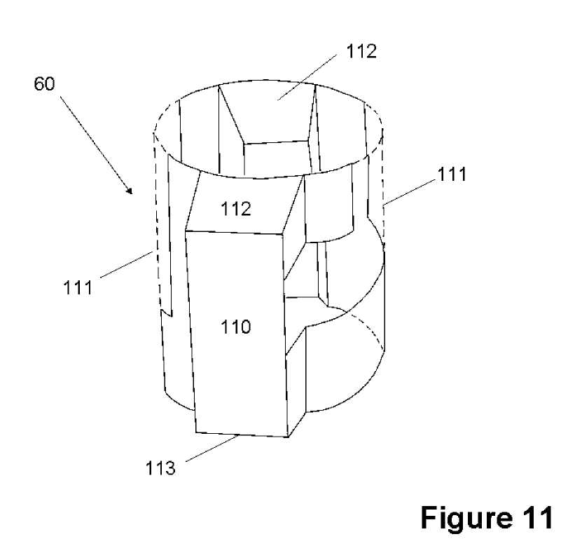

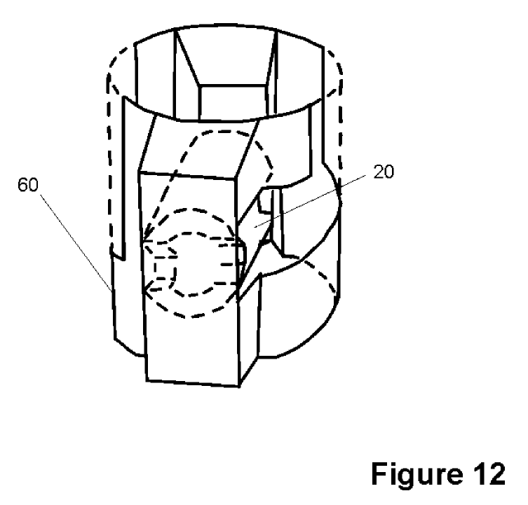

As shown in FIGS. 11-12, the rotation cannula (60) is substantially an open cylinder having a hollow rectangular cross member or, alternatively, an extension for the lateral spinous process stabilizers (110), wherein the spinous process spacer (20) is placed. The leading edge (112) of the cross member (110) is tapered to facilitate initial placement between the spinous processes (12). The trailing edge (113) is open.

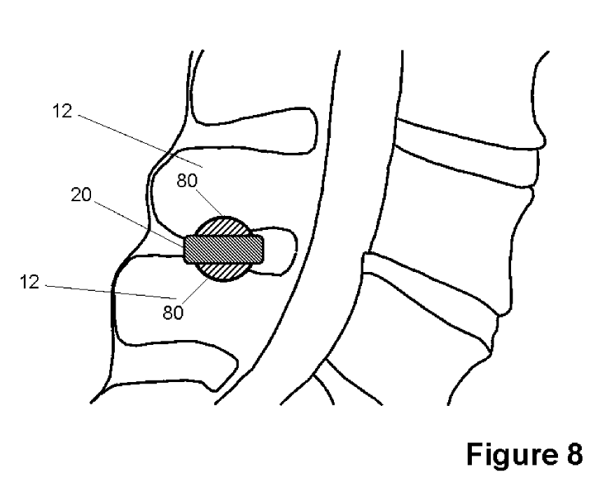

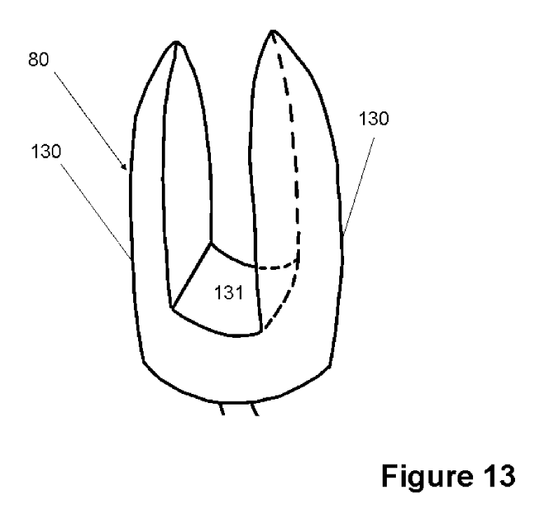

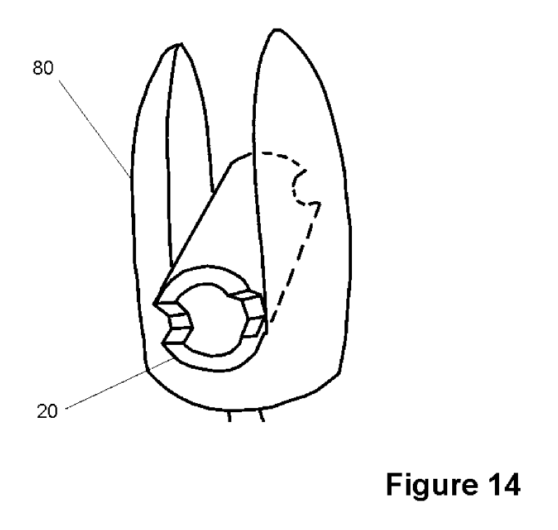

The long dimension of the rectangular cross member (110) is placed parallel to the affected spinous processes (12). With the spinous process spacer (20) saddled (131) between its prongs (130) as shown in FIGS. 13-14, the rotation instrument (80) effects placement of the spinous process spacer (20) in the rotation cannula (60) as is shown in FIG. 8. A cross section of each prong (130) flanking the spinous process spacer (20) can be seen in the figure.

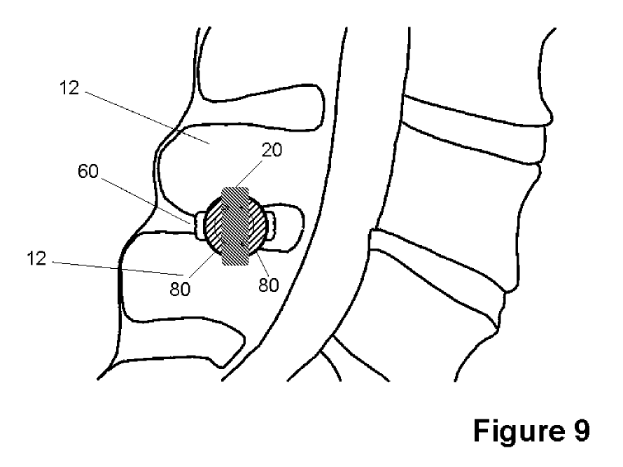

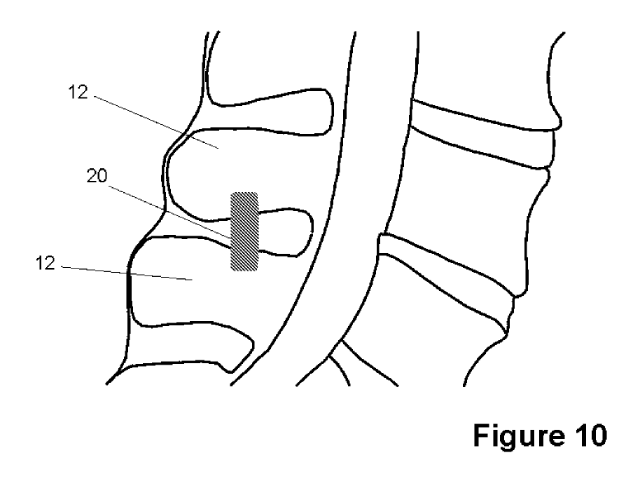

The rotation cannula (60), shown in more detail in FIGS. 11-12, is designed so that various grooves (111) allow the 90 degree rotation of the spinous process spacer (20) and removal of the rotation cannula (60) rotation instrument (80) combination leaving only the spinous process spacer (20) in place. FIG. 9 illustrates the resulting configuration after the required 90 degree rotation has been performed. Finally, FIG. 10 illustrates the placement of the spinous process spacer (20) upon removal of the cannula and rotation instrument. This lateral entry to the spinous process space combined with the 90 degree rotation of the spinous process spacer to its final position, will allow for greater preservation of the spinous process ligament and surrounding soft tissue anatomy than if the surgical approach is from a direct posterior approach.

One of the most unique features of this invention is the fusion promoting features of its design, most particularly its axial conduit (22) which not only provides a pathway wherein fusion can occur, but also provides a fillable space wherein fusion-promoting biological material can be deployed. To further this end, an additional step in the deployment technique can be used.

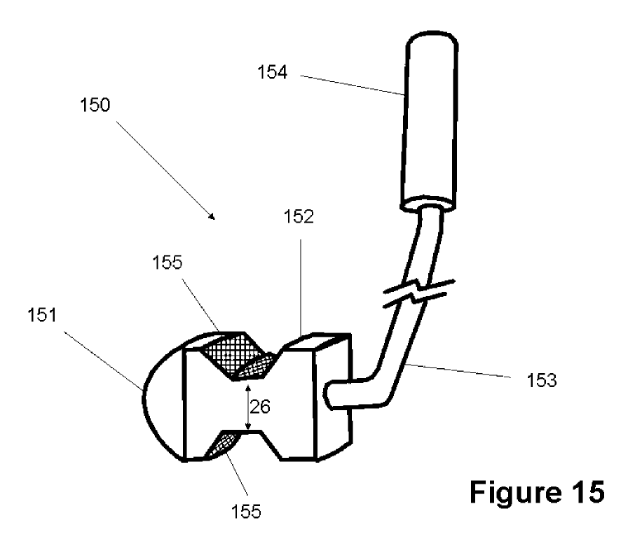

The trial rasp (150), shown in FIG. 15, is introduced into the interspinous process space (14) after distraction. Having a bullet shaped leading edge (151) to facilitate insertion, its body (152) replicates the shape of the spinous process spacer (20), particularly the dimension matching the desired interspinous process spatial distance (26).

The trial rasp (150) accomplishes several things. It tests the space for acceptance of the intended implant (20) by placing a trial in place which is representative of the size and shape of the actual implant (20). Introduction of the trial rasp (150) and particularly the rasping surface (155) causes bleeding bone and enhances bone growth and fusion. It additionally shapes and prepares the affected area of the spinous process (12) to engage and mate more intimately with the notch (23). Furthermore, it strips away soft tissue from the engagement area that might otherwise be caught between the implant (20) and the spinous process (12), thereby inhibiting fusion.

Fusion resulting between the spinous process spacer implant and the two neighboring spinous processes (12), will be much less rigid than the traditional, more invasive, posterior lateral fusion or an interbody fusion. This lessened rigidity will serve to pose less risk upon the adjacent spine motion segment and consequently less risk of a condition known as “adjacent segment disease”, a serious side effect of more traditional methods.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1-8. (canceled)

9. A method for creating and maintaining a desired space between adjacent spinous processes, the method comprising:
surgically exposing the adjacent spinous processes;
elevating the adjacent spinous processes to create a space therebetween;
placing a spinous process spacer device between the adjacent spinal processes; and
positioning a pair of notches of the device such that portions of the adjacent spinous processes are engaged therewithin to maintain the size of the desired space between the adjacent spinous processes.

10. A method as in claim 9, wherein the step of exposing the adjacent spinous processes comprises making a lateral incision to access the spinous processes.

11. A method as in claim 10, wherein the step of placing the spinous process spacer device comprises passing the spinous process spacer device laterally into the space between the adjacent spinous processes.

12. A method as in claim 9, wherein the step of placing the spinous process spacer device comprises using an implant holder to handle and secure the spinous process spacer device.

13. A method as in claim 9, further comprising attaching the spinous process spacer device to the adjacent spinous processes.

14. A method as in claim 13, wherein the spinous process spacer device is attached to the adjacent spinous processes using one of suturing and cabling.

15. A method as in claim 9, further comprising passing a trial rasp device into the space between the spinous processes prior to placement of the spinous process spacer device.

16. A method as in claim 15, wherein the trial rasp device is used to perform one of rasping the spinous processes, shaping and preparing the spinous processes to engage and mate with the notches, and stripping soft tissue from the spinous processes.

17. A method as in claim 9, further comprising filling an axial conduit of the spinous process spacer device with a bone growth-promoting material.

18. A method as in claim 9, wherein the spinous process spacer device is fabricated from a biocompatible material to promote integration of the spinous process spacer device into surrounding tissue.

19. A method as in claim 9, wherein the step of elevating the adjacent spinous processes to create a space therebetween is performed prior to placement of the spinous process spacer device between the adjacent spinous processes.

20. A method as in claim 9, wherein the step of elevating the adjacent spinous processes comprises creating the space such that the space is equal to or greater than the desired space.

21. A method for creating and maintaining a desired space between adjacent spinous processes, the method comprising:
surgically exposing the adjacent spinous processes from a lateral aspect;
placing a spinous process spacer device between the adjacent spinal processes such that a longitudinal axis of a tubular member of the device is oriented at a transverse angle with respect to the spine;
rotating the device within the mid-sagittal plane such that the longitudinal axis of the tubular member is substantially parallel to the spine; and
positioning a pair of notches of the device such that portions of the adjacent spinous processes are engaged therewithin to maintain the size of the desired space between the adjacent spinous processes.

22. A method as in claim 21, wherein the step of placing a spinous process spacer device comprises orienting the longitudinal axis of the tubular member of the device substantially at a right angle with respect to the spine.

23. A method as in claim 21, wherein the step of rotating the device comprises rotating the device approximately 90 degrees within the mid-sagittal plane.

24. A method as in claim 21, wherein the step of exposing the adjacent spinous processes comprises making a lateral incision to access the spinous processes.

25. A method as in claim 24, wherein the step of placing the spinous process spacer device comprises passing the spinous process spacer device laterally into the space between the adjacent spinous processes.

26. A method as in claim 21, further comprising distracting the adjacent spinous processes to create a space therebetween prior to placement of the spinous process spacer device between the adjacent spinous processes.

27. A method for stabilizing adjacent spinous processes, the method comprising:
accessing an interspinous space from a lateral aspect thereof to expose the adjacent spinous processes;
passing the spinous process spacer device laterally into the interspinous space between the adjacent spinous processes;
orienting the device such that a longitudinal axis of the device is substantially parallel to the spine; and
engaging opposing notches of the device with respective portions of the adjacent spinous processes to maintain the size of the desired space between the adjacent spinous processes.

28. A method as in claim 27, wherein the step of passing the spinous process spacer device into the interspinous space comprises orienting the longitudinal axis of the tubular member of the device substantially at a right angle with respect to the spine.

29. A method as in claim 27, wherein the step of orienting the device comprises rotating the device within the mid-sagittal plane.

30. A method as in claim 29, wherein the step of orienting the device further comprises rotating the device approximately 90 degrees within the mid-sagittal plane.
